(19) United States
(12) Patent Application Publication
(10) Pub. No.: US 2026/0338677 A1
(43) Pub. Date: Oct. 29, 2026

(54) SYSTEM AND METHOD FOR CONTAMINANT DETECTION IN FLUID

(71) Applicant: CYWAT TECHNOLOGIES LTD., Givat Shmuel (IL)

(72) Inventors: David FLAISHMAN, Bnei Brak (IL); Thor HALPERIN, Givat Shmuel (IL)

(73) Assignee: CYWAT TECHNOLOGIES LTD., Givat Shmuel (IL)

(21) Appl. No.: 19/700,448

(22) Filed: Jun. 8, 2026

Related U.S. Application Data
(63) Continuation-in-part of application No. PCT/IL2024/051162, filed on Dec. 8, 2024.
(60) Provisional application No. 63/608,267, filed on Dec. 10, 2023.

(51) Int. Cl. G01N 21/25; G01N 21/31; G01N 21/94; G01N 33/18

(52) U.S. Cl. CPC G01N 21/255; G01N 21/31; G01N 21/94; G01N 33/18

(57) ABSTRACT

A system and method for detecting and identifying contaminants in fluid comprising at least one light source configured for illuminating a fluid sample, at least one optical detector configured for detecting light reemitted by the fluid sample, wherein the at least one optical detector is set at various angles to the at least one light source, and a processor configured for processing measured spectrograms, wherein the processor includes an artificial intelligence algorithm (AI) configured to process and analyze the measured spectrograms, and wherein the at least one optical detector and/or the at least one light source are stationary and/or rotating relative to the sample.

### RELATED APPLICATIONS

This application is a Continuation in Part of International patent application no. PCT/IL2024/051162 filed on 8 Dec. 2024, which claims the benefit of priority under 35 USC § 119(e) of U.S. Provisional Patent Application No. 63/608,267 filed on 10 Dec. 2023, the contents of which are incorporated herein by reference in their entirety.

### FIELD AND BACKGROUND OF THE INVENTION

The present invention, in some embodiments thereof, relates to a system and method for detecting substances in fluid, particularly, but not exclusively for detecting and identifying low concentrations of contaminants in water.

The U.S. EPA has set standards for more than 80 contaminants that may occur in drinking water and pose a risk to human health. These contaminants can be divided into two groups based on the health effects they cause: acute and chronic.

Acute health effects can occur within hours or days of exposure to a contaminant. Such effects can be caused by almost any contaminant, if exposure levels are high enough, but microbes such as bacteria and viruses are the most likely to cause acute health effects in drinking water. Microbial contaminants can make people sick and can be dangerous or deadly for people with weakened immune systems.

Chronic health effects can occur after long-term exposure to a contaminant. Drinking water contaminants that can have chronic effects include chemicals, radionuclides, and minerals. Examples of chronic health effects include cancer, liver or kidney problems, and reproductive difficulties.

Therefore, it is vital to monitor the levels of contaminants, particularly low concentrations of contaminants in “clean” water. Monitoring can provide a means of detecting and predicting concentrations of contaminants which can have adverse effects on health.

Currently, there is no reliable way to detect and identify low concentration contaminants in water, such as tap water.

International patent application No. 2021/024214 appears to disclose an apparatus for generating a two-dimensional map representative of a turbid or clear medium includes a system for generating incoherent light within a medium, a light collecting system that is movable or stationary relative to the medium being analyzed and that is arranged for collecting light exiting the medium, and a spectrum analyzer configured to determine spectrum data of the light exiting the turbid medium and to transmit the spectrum data to a computing unit configured to generate a two-dimensional map, in which one dimension of the map is wavelength and a second dimension is a position of the light collecting system. The invention is also directed to a method for classifying media using the two-dimensional map generated with the apparatus. The method comprises steps of feeding and training neural networks and using the trained neural networks to classify unknown media.

Additional background art includes U.S. patent application No. 20140118525, U.S. patent application No. 20220283083, U.S. patent application No. 20100233329, U.S. Pat. Nos. 5,867,276, 9,014,430, U.S. patent application No. 2019003976, U.S. patent application No. 2019033132, Chinese patent No. 2720434, and U.S. Pat. No. 10,746,680.

Therefore, there is still a need for a system and a method to detect and identify low concentrations of contaminants in water.

### SUMMARY OF THE INVENTION

According to an aspect of some embodiments of the invention, there is provided a method for detecting and identifying contaminants in fluid, the method including: measuring control spectral refractions of a plurality of control media with known contents the control spectral refractions including multiple angles and/or wavelengths; selecting one of the plurality of control media as a base media and labelling a corresponding spectral reflection as a base spectral reflection; measuring a test spectral refraction of a test medium including the multiple angles and/or wavelengths; determining a test ratio of a magnitude of the test spectral refractions to the base spectral reflection from the multiple angles and/or wavelengths; and comparing the test ratio to ratios of a magnitudes of a plurality of the control spectral refractions to the base spectral refraction to determine the contents of the test medium.

According to some embodiments of the invention, the method further includes mapping of the test spectral refraction and the base spectral reflection.

According to some embodiments of the invention, the method further determining a ratio of the mapped test spectral refraction to the base spectral reflection.

According to some embodiments of the invention, the method further includes comparing the test ratio of the mapped spectral refractions to mapped spectral refractions to ratios of the control spectra in a database.

According to some embodiments of the invention, the map is a three-dimensional spectral map wherein a first axis is refraction angle, a second axis is wavelength, and a third axis is intensity.

According to some embodiments of the invention, the map is a two-dimensional spectral map wherein a first axis is refraction angle, a second axis is wavelength.

According to some embodiments of the invention, the map is a two-dimensional spectral map wherein a first axis is refraction angle, a second axis is intensity.

According to some embodiments of the invention, the method further includes collecting the spectral refraction by at least one detector.

According to some embodiments of the invention, the collecting is by at least one detector which is stationary relative to the test medium.

According to some embodiments of the invention, the collecting is by at least one detector which is rotating relative to the test medium.

According to some embodiments of the invention, the method further includes at least one light source configured for illuminating the sample.

According to some embodiments of the invention, the illuminating is by at least one light source which is stationary relative to the test medium.

According to some embodiments of the invention, the illuminating is by at least one light source which is rotating relative to the test medium.

According to some embodiments of the invention, where the comparing is via an artificial intelligence algorithm (AI) is configured to process and analyze spectral refractions.

According to an aspect of some embodiments of the invention, there is provided a system for detecting and identifying contaminants in fluid, including: at least one light source configured for illuminating a fluid sample; at least one optical detector configured for detecting light refracted by the fluid sample, wherein the at least one optical detector is configured to detect light passing through the fluid sample at various angles to the at least one light source; and a processor configured for collecting and processing measured spectral data for different wavelengths, different relative angles or both.

According to some embodiments of the invention, the processor is configured to control the relative angle between the light source and the detector.

According to some embodiments of the invention, the processor is configured to control the relative angle between the light source and the detector by: a) selecting multiple stationary light sources and detectors; or b) moving a detector or light source with an actuator; or c) measuring refracted light from a stationary or moving light source at different angles with multiple stationary or moving detectors simultaneously; or d) any combination of steps a, b and c.

According to some embodiments of the invention, the system further includes a database connected to the processor.

According to some embodiments of the invention, the system further includes a user interface.

According to some embodiments of the invention, the user interface includes a display.

According to some embodiments of the invention, the at least one light source is stationary relative to the fluid sample.

According to some embodiments of the invention, the at least one light source is rotating relative to the sample.

According to some embodiments of the invention, the at least one detector is stationary relative to the fluid sample.

According to some embodiments of the invention, the at least one detector is rotating relative to the fluid sample.

According to some embodiments of the invention, the processor includes an artificial intelligence algorithm (AI) configured to process and analyze measured refraction spectra.

According to some embodiments of the invention, the processor is further configured for: measuring control spectral refractions of a plurality of control media with known contents the control spectral refractions including multiple angles and/or wavelengths; selecting one of the plurality of control media as a base media and labelling a corresponding spectral reflection as a base spectral reflection; measuring a test spectral refraction of a test medium including the multiple angles and/or wavelengths; determining a test ratio of a magnitude of the test spectral refractions to the base spectral reflection from the multiple angles and/or wavelengths; and comparing the test ratio to ratios of a magnitudes of a plurality of the control spectral refractions to the base spectral refraction to determine the contents of the test medium, and comparing the test ratio to ratios of a magnitudes of a plurality of the control spectral refractions to the base spectral refraction to determine the contents of the test medium.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-ID: Block diagrams of systems for detecting and identifying contaminants in fluid, in accordance with some embodiments.

FIG. 2: A flow chart of a method for detecting and identifying contaminants in fluid, in accordance with some embodiments.

FIGS. 3A-3H: Exemplary three-dimensional map of angle vs. wavelength vs. intensity for several water samples, optionally spiked with various contaminants, in accordance with some embodiments.

FIG. 4: Exemplary map of wavelength vs. intensity for a water sample, in accordance with some embodiments.

FIG. 5: Exemplary map of wavelength vs. intensity ratio for a water sample spiked with 1 ppm manganese compared to that of a database clean water sample, in accordance with some embodiments.

FIG. 6: Exemplary map of wavelength vs. intensity or a clean water sample compared to that of a database clean water sample, in accordance with some embodiments.

FIGS. 7A-B: Exemplary chart of F1-score and false positive and negative rates, respectively, in accordance with some embodiments.

FIG. 8A illustrates Table 1 of training parameters.

FIG. 8B illustrates Table 2 of experimental results.

### DESCRIPTION OF SPECIFIC EMBODIMENTS

The present invention, in some embodiments thereof, relates to a system and method for detecting substances in fluid, particularly, but not exclusively for detecting and identifying low concentrations of contaminants in water.

### Overview

Some embodiments relate to a system and method for detecting contaminants in fluid. Optionally, for detecting and/or identifying low concentrations of contaminants in a water source (e.g., tap water, rivers, lakes, etc.). Optionally, the system and method may use spectral methods for detecting and/or identifying low concentrations of contaminants in a fluid sample. Alternatively, the system may be used to identify substances in other fluid samples. For example, fluids may include gases (e.g., air, oxygen, nitrogen, argon, methane etc.) and/or liquids (e.g., water, ethanol, benzene, chloroform, acetic acid, ammonia, bleach, etc.) and/or combinations thereof.

Advantageously, the system and method provide a means for detecting and/or identifying low concentrations of contaminants in a fluid sample. Additionally, or alternatively, the system and method provide a means of monitoring water quality. Additionally, or alternatively, the system and method provide a means of identifying and/or predicting trends in the water quality of a particular water source.

According to some embodiments, the contaminants may be pathogens, such as bacteria, viruses and/or parasites. Non-limiting examples of pathogens include cryptosporidium, Campylobacter, E. coli, Salmonella typhi, Salmonella, Shigella, Legionella, Vibrio vulnificus, Campylobacter, Giardia lamblia, Hepatitis A, Calicivirus (e.g., Norwalk- and Sapporo-like viruses), Enterovirus (e.g., polio, echo, encephalitis, conjunctivitis, and Coxsackie viruses]), Norovirus, Rotavirus, SARS-type viruses, etc. and/or any combination thereof.

According to some embodiments, the contaminants may be organic and/or inorganic chemicals. Non-limiting examples of such contaminants include hormones, plastics, their derivatives and decomposition products, nitrates, fluoride, phosphates, metals (e.g., copper, manganese, iron, arsenic, etc.), acids, bleach, salts, pesticides, etc.

According to some embodiments, the system and method may measure the optical properties of a fluid sample. Optionally, the system and method may measure the spectroscopic properties of a fluid sample. Optionally, the fluid sample may be clear and/or turbid.

According to some embodiments, the method may include the following steps:
I. directing light from one or more light sources into a sample;
II. detecting the light exiting the sample with one or more optical detectors; and
III. analyzing the detected light with a spectrometric system.

In some embodiments, the sample, may include a fluid. For example, the sample may include water with one or more contaminants. Optionally, a base sample is selected, for example, the base sample may be clean water and/or water with contaminated with contaminants (for example, the base sample may be the expected contents and/or desired contents of source to be tested). Optionally, control spectra are measured from the control samples of the base sample and a known further contaminant are tested. For example, measuring a spectrum may include measuring light at refracted at several angles with respect to the source of the light. The light may be measured and/or projected at one or more frequencies.

According to some embodiments, a spectrum may be measured with one or more light sources that may be directed at the sample at multiple angles while the one or more optical detectors are stationary. According to some embodiments, one or more light sources may be directed at the fluid sample while the one or more optical detectors may measure light from various angles. For example, a light source and/or a detector may be rotated around the sample. According to some embodiments, one or more light sources may be stationary with respect to the fluid sample while the one or more optical detectors are stationary and/or rotating. According to some embodiments, one or more light sources may be stationary with respect to the fluid sample while the one or more optical detectors are rotating. According to some embodiments, one or more light sources may be stationary and/or rotating with respect to the fluid sample while the one or more optical detectors are stationary and/or rotating.

According to some embodiments, the wavelength of the light from each of the one or more light sources may have the same wavelength. Optionally, the wavelength of the light from each of the one or more light sources may have the different wavelengths. Optionally, the one or more light sources may be activated simultaneously and/or sequentially.

According to some embodiments, the light may be incoherent and/or coherent. Optionally, the light may have a wavelength in the range between about 100 to about 400 nm (ultraviolet), and/or between about 400 to about 800 nm (visible), and/or between about 800 nm to about 1 mm (infrared), and/or about 1 mm to about mm, and/or any sub-range therebetween.

According to some embodiments, light may be irradiated from one direction and an optical detection system may measure the light intensity from multiple exit directions from the fluid sample.

According to some embodiments, one or more optical detectors may be rotated around the fluid sample, such that spectrograms may be recorded from multiple angles. According to some embodiments, the one or more optical detectors may be moved linearly. According to some embodiments, the one or more optical detectors may be moved non-linearly. According to some embodiments, the one or more optical detectors may be located at multiple stationary positions around the fluid sample, such that spectrograms may be recorded from multiple angles. Optionally, the light exiting the fluid sample detected by the one or more optical detectors may have multiple wavelengths. Optionally, the light exiting the test sample may have different wavelengths depending on the angle and/or position of the one or more detectors. Optionally, the one or more optical detectors may be spectrometers. Optionally, the spectrometer may be configured to determine spectral data of the reemitted light.

According to some embodiments, the spectrograms detected by one or more optical detectors may be recorded in a processing unit. Optionally, the processing unit may include one or more algorithms and/or an artificial intelligence algorithm (AI) configured to process and/or analyze the recorded data.

According to some embodiments, the shape of the container holding the test sample may be designed to avoid optical aberrations. Additionally, or alternatively, optical aberration correction may be applied by the processing unit.

According to some embodiments, recording spectrograms at multiple angles between the light source and the detector may provide the angular dependence of the optical properties of the test sample. For example, refracted light may be measured at 2, between 3 to 5, between 6 to 10, between 11 to 18 and/or more than 18 different angles between the light source and the sensor. Optionally, the processor may use the spectrograms obtained at different angles to generate a two-dimensional spectral map. Optionally, in the multi-dimensional map one axis may be the wavelength and the other axes may be the collecting angle and intensity. Optionally, the processor may use the spectrograms obtained at different angles to generate a three-dimensional spectral map. Optionally, in the three-dimensional map, a first axis may be the wavelength, a second axis may be the collecting angle, and a third axis may be the intensity. Optionally, a set of multi-dimensional dependent variables (e.g., angle, wavelength, intensity, etc. and any combination thereof) may be derived from each two-dimensional map and/or three-dimensional spectral map.

According to some embodiments, the two-dimensional map and/or three-dimensional spectral map of one or more standard samples may be collected prior to measurement of a test sample. Optionally the two-dimensional map and/or multi-dimensional spectral map of one or more standard samples may be stored in a database. Optionally, the base sample may be a sample of the clean fluid e.g., clean water, distilled water, deionized water, pure water, etc.

According to some embodiments, a spectrum with one or more multi-dimensional dependent variables of a base sample (e.g., pure water) may be subtracted from each set of values from the multi-dimensional dependent variables measured in a test sample. Additionally, or alternatively, each set of values from the multi-dimensional dependent variables measured in a test sample may be divided by one or more multi-dimensional dependent variables of a standard sample (e.g., pure water). Optionally, characterization of the contaminate within the test sample may be based on the ratio between the reference and measured two-dimensional map and/or multi-dimensional spectral map.

According to some embodiments, these results may be analyzed by a trained artificial intelligence algorithm, linear fitting, etc. Optionally, these results may be compared to a set of values measured for spectra of know concentrations of common contaminants in control samples (e.g., in a database). Optionally, if the set of standard contaminants does not give a good fit, then this may be an indication of the presence of an unusual contaminant.

According to some embodiments, the processing unit may include a spectral database. Optionally, the spectral database may include one or more two-dimensional maps and/or three-dimensional spectral maps from fluid samples spiked with various contaminants in various concentrations. Optionally, the measured two-dimensional map and/or three-dimensional spectral map may be compared to the two-dimensional map and/or three-dimensional spectral map in the spectral database to determine the presence of a contaminant. Optionally, the measured two-dimensional map and/or three-dimensional spectral map may be compared to the spectrograms in the spectral database to identify a contaminant present in the fluid sample. Optionally, the measured two-dimensional map and/or three-dimensional spectral map may be compared to the spectrograms in the spectral database to determine the concentration of a detected and/or identified contaminant.

According to some embodiments, the light intensity of the various wavelengths through the measured test sample materials may be used to detect and/or identify unknown contaminants in a test sample.

According to some embodiments, comparison of the measured and database two-dimensional map and/or three-dimensional spectral map may be performed by applying a trained artificial intelligence (AI) algorithm. Optionally, the trained AI algorithm may be trained on a plurality of labeled two-dimensional maps and/or three-dimensional spectral maps. Optionally, the labeling may be manual. Optionally, samples of a base media may be labeled as “clean”. Optionally, control samples containing a known amount of a specific contaminant may be labeled by the contaminant type, regardless of the concentration. Optionally, the labeling may indicate the type of impurity and/or the concentration of the impurity. Optionally, the training may be supervised and/or unsupervised.

According to some embodiments, an AI algorithm may be applied to a series of two-dimensional maps and/or three-dimensional spectral maps of test samples taken over a specific time period from a water source. Optionally, the AI algorithm may detect and/or predict a trend in the concentration of one or more contaminants in the test samples based on a series of two-dimensional maps and/or three-dimensional spectral maps taken over a specific time period from the same water source. Optionally, the time period may be minutes, hours, days, weeks, months and/or years. Optionally, the AI algorithm may be trained to identify a source of one or more contaminants e.g., by tracking the age of the pipes, industries in the area of the water source and/or water table, weather patterns, population growth, changes in health of the population, changes in pesticides and/or herbicides used, etc.

According to some embodiments, an AI algorithm may include one or more of the following elements: 1. prediction networks. 2. sample processing methodology; 3. training methodology; 4. normalization; 5. cleaning the datasets; 6. visualization. Optionally, each element may consist of several aspects. For example, when choosing the prediction network, a user and/or AI may decide on the type of the network, and/or the output layer, and/or the meta parameters. Optionally, to increase the accuracy of the prediction multiple experiments may be run in order to select the optimal solutions.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

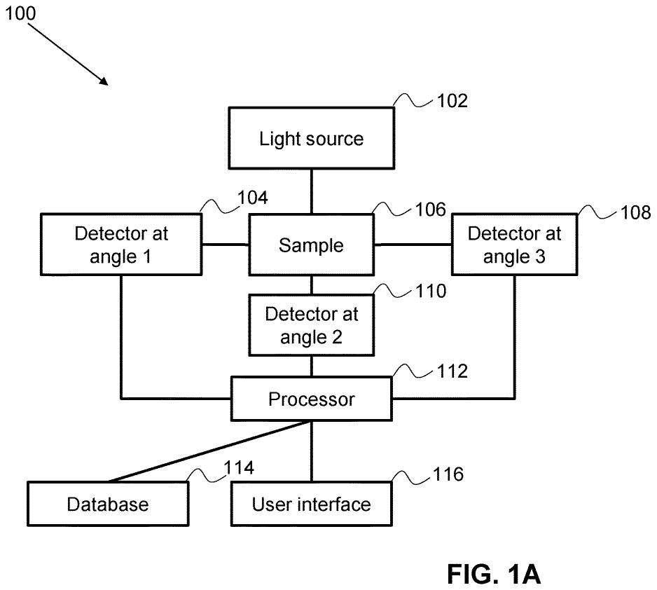

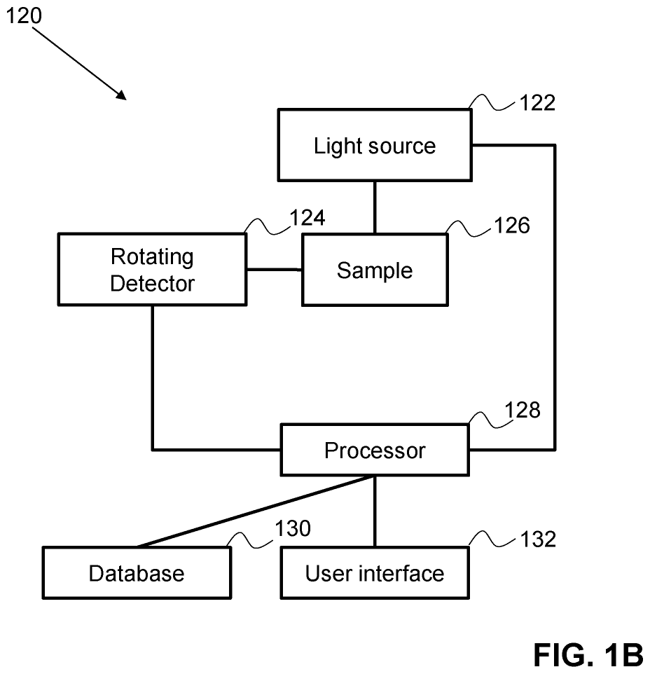

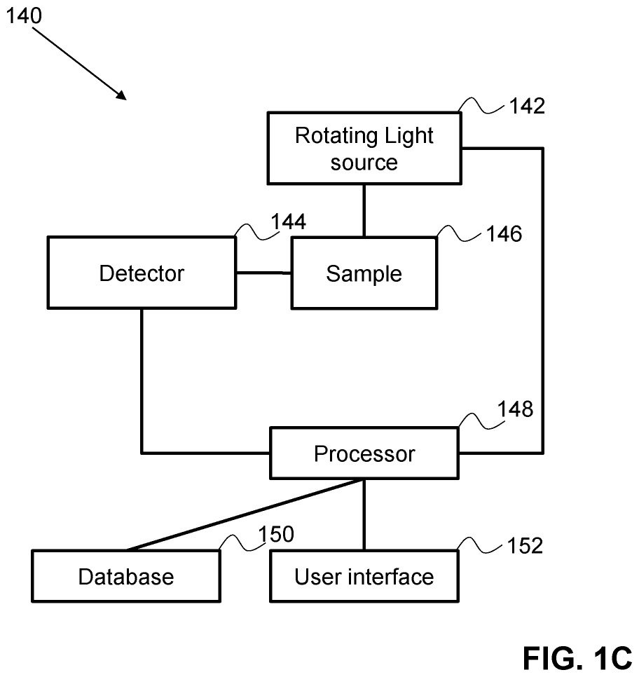

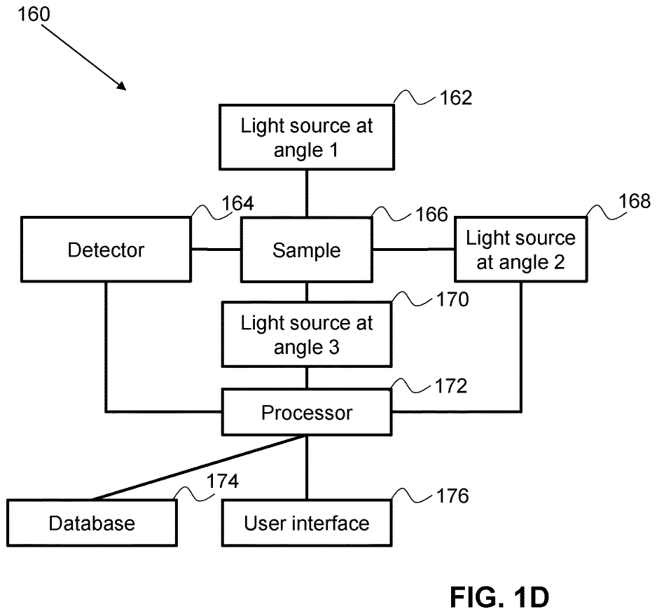

FIG. 1A is a block diagram of a system for detecting and identifying contaminants in fluid, in accordance with some embodiments. For example, system 100 may include a light source 102, which may irradiate a sample 106. Reemitted light may be detected by one or more optical detectors, set at various angles to the light source (e.g., a first optical detector 104, a second optical detector 108, a third optical detector 110, etc.). The measured spectrograms may be processed by a processor 112. A database 114 may be connected to processor 112. Additionally, the system may include a user interface 116. Optionally, user interface 116 may include a display.

FIG. 1B is a block diagram of a system for detecting and identifying contaminants in fluid, in accordance with some embodiments. For example, system 120 may include a light source 122, which may irradiate a sample 126. Reemitted light may be detected by a rotating optical detector 124 that moves to various angles to the light source 122. The measured spectrograms may be processed by processor 128. A database 130 may be connected to processor 128. Additionally, the system may include a user interface 132. Optionally, user interface 132 may include a display.

FIG. 1C is a block diagram of a system for detecting and identifying contaminants in fluid, in accordance with some embodiments. For example, system 140 may include a rotating light source 142, which may irradiate sample 146 at various angles to an optical detector 144. Reemitted light may be detected by optical detector 144. The measured spectrograms may be processed by a processor 148. A database 150 may be connected to processor 148. Additionally, user interface 152 may be connected to a user.

FIG. 1D is a block diagram of a system for detecting and identifying contaminants in fluid, in accordance with some embodiments. For example, system 160 may include multiple light sources (e.g., a first light source 162, a second light source 168, a third light source 170, etc.), which may irradiate sample 166 at various angles to an optical detector 164. Reemitted light may be detected by the optical detector 164. The measured spectrograms may be processed by a processor 172. A database 174 may be connected to processor 172. Additionally, a user interface 176 may be connected to a user.

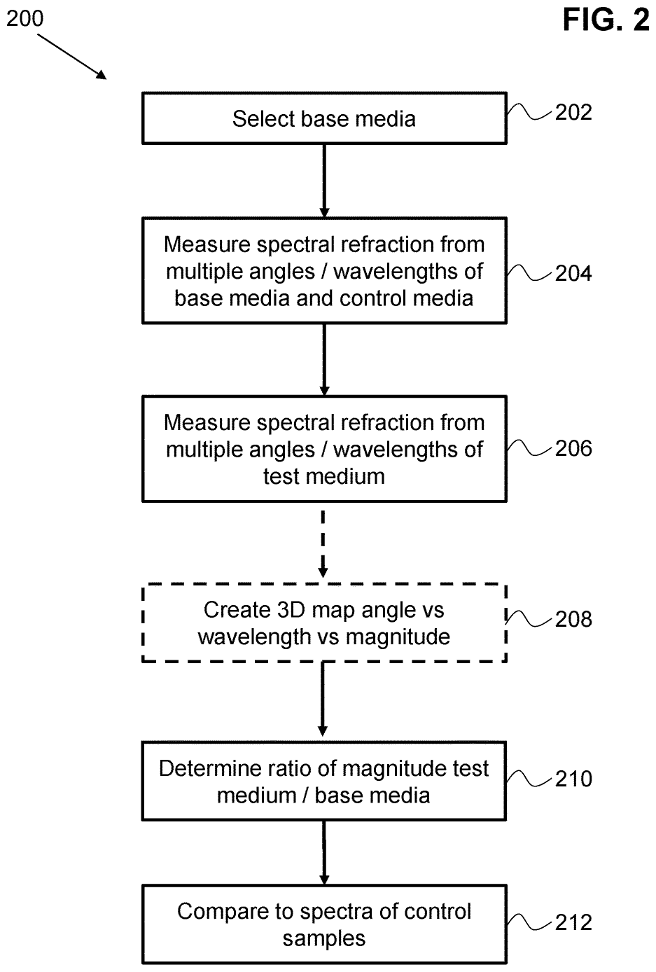

FIG. 2 is a flow chart of a method for detecting and identifying contaminants in fluid, in accordance with some embodiments. For example, in method 200, known sample/s are labeled 202 as base media. The spectral refraction from multiple angles and/or wavelengths of the base media and/or the control samples of the base media with one or more known contaminants are measured 204. Then the spectral refraction from multiple angles and/or wavelengths of a test medium is measured 206. A three-dimensional map of the angle, wavelength and intensity is optionally created 208. The ratio of the magnitude of the test medium to the base media is determined 210 and compared 212 to the control samples, e.g., in a database.

In some embodiments, the base sample and/or control samples may include different base fluids. Optionally, a correction may be made to normalize the control samples to a different base fluid.

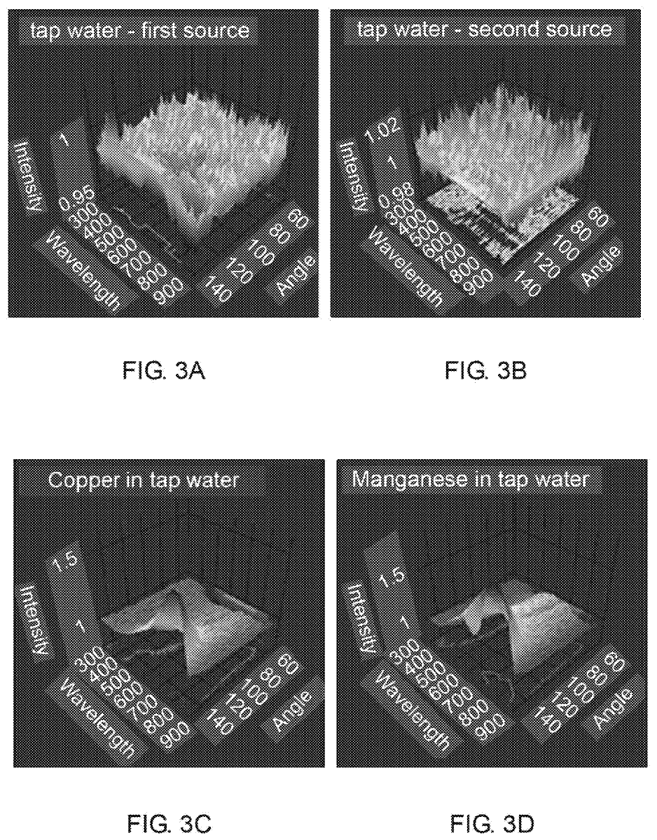

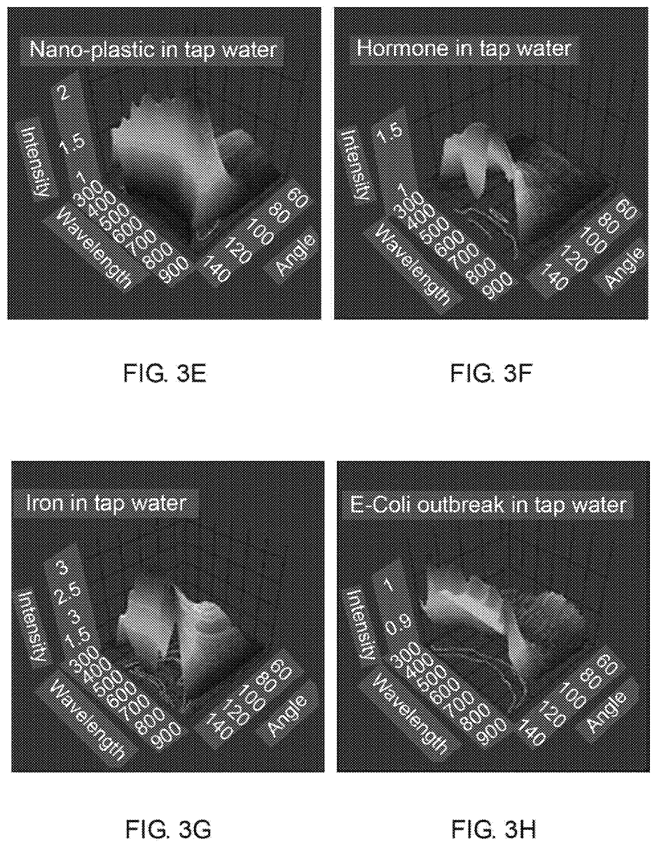

FIG. 3A-3H are exemplary three-dimensional maps of angle vs. wavelength vs. intensity for several water samples, optionally spiked with various contaminants, in accordance with some embodiments. For example, in each of the exemplary three-dimensional maps the intensity (ratio), wavelength [nm], and angle [deg] are displayed. FIGS. 3A and 3B show exemplary three-dimensional maps of samples of tap water; FIG. 3C shows an exemplary three-dimensional map of a sample of tap water containing copper; FIG. 3D shows an exemplary three-dimensional map of manganese in tap water; FIG. 3E shows an exemplary three-dimensional map of Nano-Plastic in tap water; FIG. 3F shows an exemplary three-dimensional map of a hormone in tap water; FIG. 3G shows an exemplary three-dimensional map of an Iron in tap water; and FIG. 3H shows an exemplary three-dimensional map of E-coli outbreak tap water.

According to some embodiments, the system may facilitate real-time detection of low concentrations of contaminants in a water source, moreover, the system may facilitate identification of the contaminant and/or the concentration thereof, e.g., if the contaminant is chemical, bacterial, an industrial pollutant, etc.

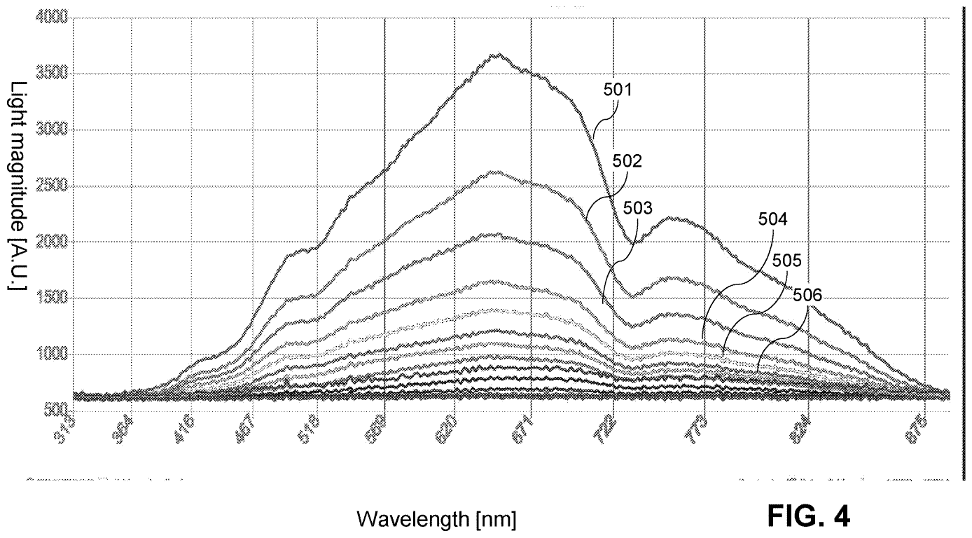

FIG. 4 is an exemplary two-dimensional map of wavelength vs. magnitude for a water sample, in accordance with some embodiments. The various lines of the graphs represent the different angles e.g., 135° 501, 130° 502, 125° 503, 120° 504, 115° 505, 110° 506, between the light source and the one or more optical detectors. The lower lines are steeper angles.

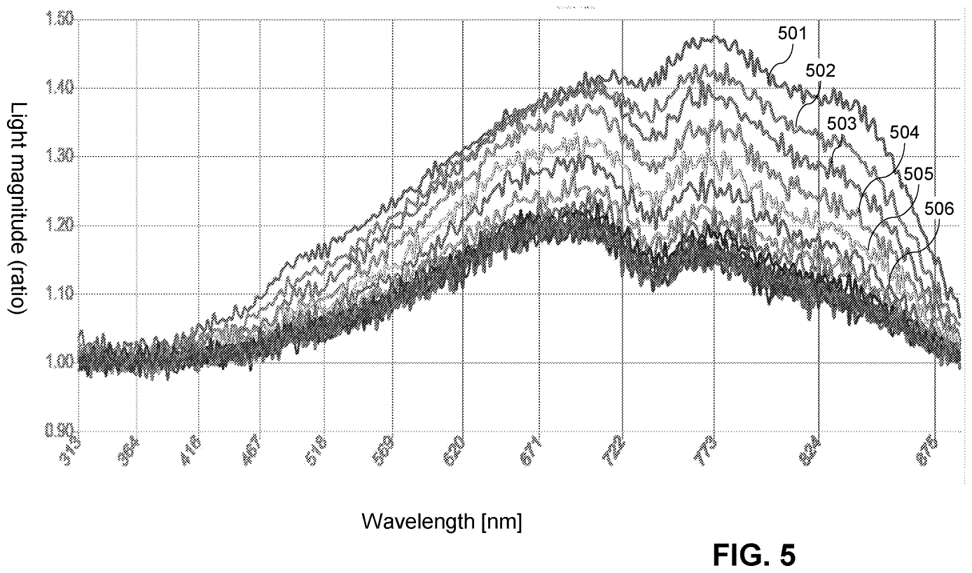

FIG. 5 is an exemplary two-dimensional chart of ratio magnitude vs. wavelength at various angles for 1 ppm manganese acetate divided by magnitude of a clean water sample at the same angle and wavelength, in accordance with some embodiments. The various lines of the graphs represent the different angles e.g., 135° 501, 130° 502, 125° 503, 120° 504, 115° 505, 110° 506, between the light source and the one or more optical detectors. The ratio of light magnitude of the test sample is shown relative to clean water. It is seen that by normalizing with the clean water magnitudes, the various angles show a consistent pattern that makes it easier to determine the contaminant.

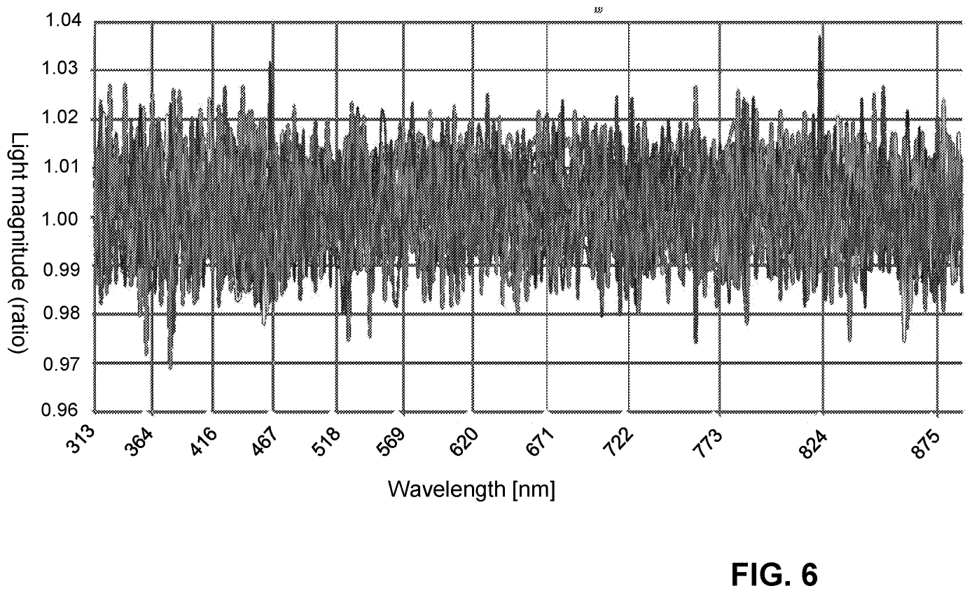

FIG. 6 is an exemplary two-dimensional chart of wavelength vs. magnitude for a clean water sample compared to that of a database clean water sample, in accordance with some embodiments. The various lines of the graphs represent the different angles between the light source and the one or more optical detectors. The ratio of light magnitude of the clean water sample is shown relative to reference clean water. It can be seen that the clean water normalized by clean water produces and easily recognizable straight-line signal at various angles and wavelengths. Thus, any contaminant that causes deviation from the straight-line behavior is easily detected.

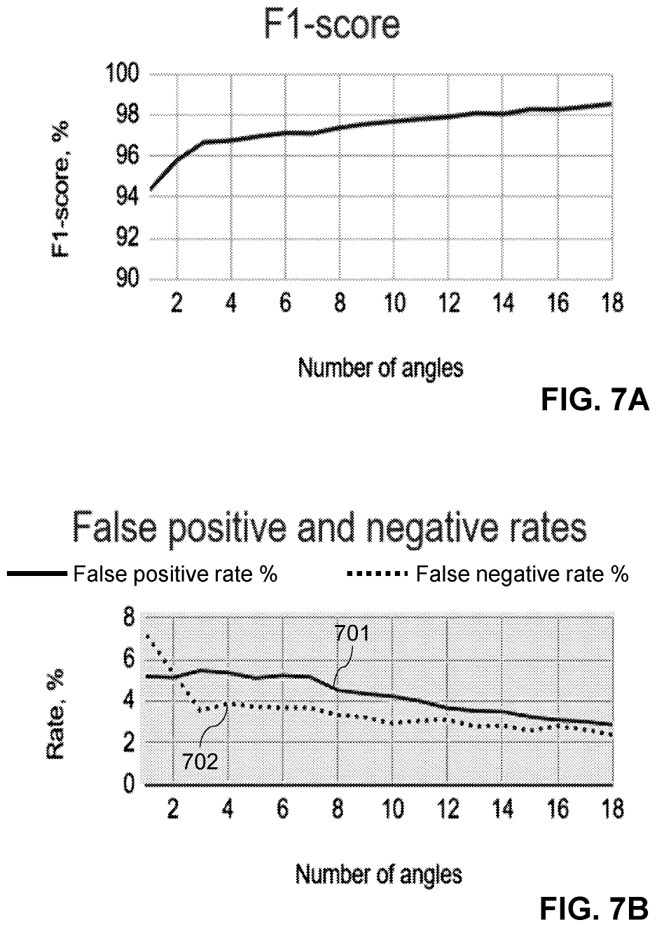

FIGS. 7A-B are exemplary charts of F1-score and false positive and negative rates, respectively, in accordance with some embodiments. For example, the F1-score is the normalization per scan, wherein the algorithm removes the median and scales the data according to the quantile range between 0.1% and 99.9%. The F1-score increases as the number of detection angles increases. The percentage of false positives 701 and false negatives 702 is calculated by the AI. The rate of the percentages of false positives 701 and false negatives 702 decreases as the number of detection angles increases.

These embodiments are provided by way of example and are in no means intended to limit the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1: Development Process

### Methodology for Contaminators Identification in Water

Machine learning techniques were used in order to identify contaminants in a base media. The base media was selected as water. In particular, the convolutional neural network (CNN) was used. CNN was found to be suitable for recognizing contaminants. The CNN included a class of deep neural networks, commonly applied to analyzing visual imagery.

A CNN was trained to identify whether a water sample includes a contaminant based on a methodology for collecting and labeling samples, and spectrographic data.

### Training and Testing Samples

The training was performed on control samples including, base media, clean water samples and water containing known contaminants. Samples containing contaminants in water were prepared. For each contaminant several concentration levels were prepared. The samples were labeled by the contaminant type, regardless of the concentration. Samples from various water sources were collected.

The AI was trained using these samples. For the training, at least 100 samples of each type of contaminant were used and at least 20 samples were tested.

Samples of water containing contaminants from a few sources were evaluated and tested.

### Normalization

Normalization was performed per scan. The average value was calculated for every scan. This algorithm removed the median, and scaled the data according to the quantile range between 0.1% and 99.9%. Centering and scaling were performed by computing the relevant statistics on each scan.

The centering and scaling statistics of the scaler were based on percentiles in order to reduce influence by marginal outliers, e.g., few numbers of very large marginal outliers.

The normalization was evaluated with different percentiles. Percentiles 0.1-99.9% yielded the highest F1 score. The F1 score is the harmonic mean of precision and recall in a single metric. Precision being the proportion of positive identifications that were actually correct and recall being the proportion of actual positive cases that were correctly identified.

### The CNN Structure

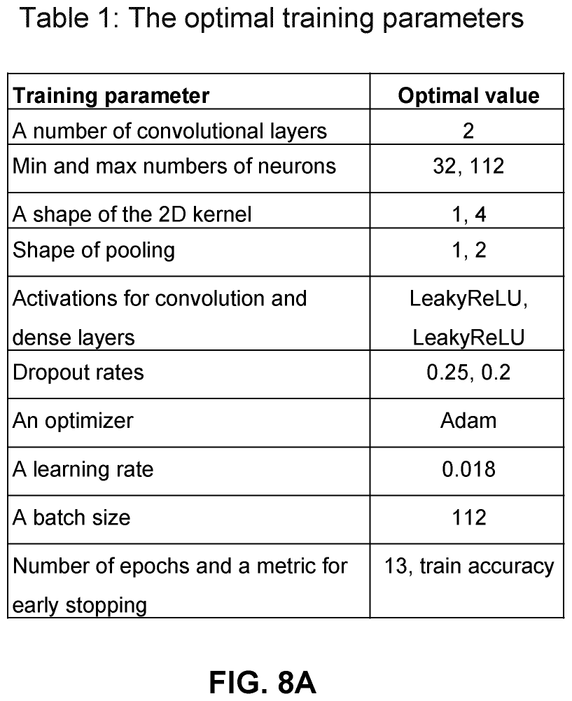

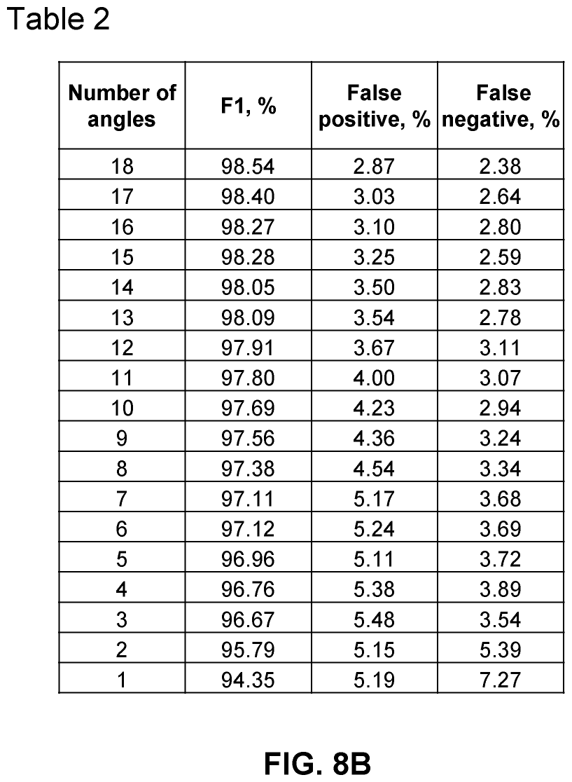

The optimal hyperparameters of the network were determined through experiments on datasets of various machines (see Table 1 of FIG. 8A). Hyperparameter tuning was performed using only the training dataset for each machine. The common hyperparameters across all machines and for the experiment were chosen based on intuition and the optimal hyperparameters for each individual machine.

In the output layer included one node for each of 10 contaminators. No nodes for deionized water (DI) or water were used.

### Model: “Sequential”

| Layer (type) | Output Shape | Param # |
|---|---|---|
| conv2d (Conv2D) | (None, 18, 98, 32) | 288 |
| leaky_re_lu (LeakyReLU) | (None, 18, 98, 32) | 0 |
| max_pooling2d (MaxPooling2D) | (None, 9, 49, 32) | 0 |
| conv2d_1 (Conv2D) | (None, 8, 46, 69) | 17733 |
| leaky_re_lu_1 (LeakyReLU) | (None, 8, 46, 69) | 0 |
| max_pooling2d_1 (MaxPooling2D) | (None, 4, 23, 69) | 0 |
| dropout (Dropout) | (None, 4, 23, 69) | 0 |
| flatten (Flatten) | (None, 6348) | 0 |
| dense (Dense) | (None, 112) | 711088 |
| leaky_re_lu_2 (LeakyReLU) | (None, 112) | 0 |
| dropout_1 (Dropout) | (None, 112) | 0 |
| dense_1 (Dense) | (None, 10) | 1130 |
| activation (Activation) | (None, 10) | 0 |

Total params: 730,239
Trainable params: 730,239
Non-trainable params: 0

### Evaluation

The experiment was carried out for 10 contaminants. For each contaminant 100 samples were collected for training and testing, and 20 samples were evaluated from different batches. An F1-score was used as the main metric, additionally false positives and missed classifications were evaluated. It was assumed that increasing the number of angles would increase the F1-score and decrease the number of false positives and false negatives.

Spectral refractions of the samples were scanned using up to 18 angles. The number of angles was varied between 1 and 18 (see FIGS. 7A and 7B). There were 153 combinations of pairs of angles, as well as 153 combinations of 16 (of 18 tested) angles. The number of possible combinations of any number of angles between 3 and 15 was significantly higher. For instances with more than 200 combinations, only 200 random combinations were evaluated to expedite the experiment. The reported metrics represent the average across the evaluated combinations of angles.

As can be seen in Table 2 of FIG. 8B, the F1-score varied between 94.35% and 98.54%.

### General

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

The terms “spectra” and “spectrograms” are used interchangeably, and as used herein refer to measurement and interpretation of the electromagnetic spectra that result from the interaction between electromagnetic radiation and matter as a function of the wavelength or frequency of the radiation.

The term “multiple” as used herein refers to 2, 3, 4, 5, 10, 12, 15, 20, or more.

The term “plurality” as used herein refers to 100, 500, 1000, 10000, 100000, 1000000, or more.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are Optionally, provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or m connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Data and/or program code may be accessed and/or shared over a network, for example the Internet. For example, data may be shared and/or accessed using a social network. A processor may include remote processing capabilities for example available over a network (e.g., the Internet). For example, resources may be accessed via cloud computing. The term “cloud computing” refers to the use of computational resources that are available remotely over a public network, such as the internet, and that may be provided for example at a low cost and/or on an hourly basis. Any virtual or physical computer that is in electronic communication with such a public network could potentially be available as a computational resource. To provide computational resources via the cloud network on a secure basis, computers that access the cloud network may employ standard security encryption protocols such as SSL and PGP, which are well known in the industry.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

As used herein the term “about” refers to ±10% The terms “comprises”, “comprising”, “includes”, “including”, “having” and their conjugates mean “including but not limited to”. The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for detecting and identifying substances in fluid, the method comprising:
measuring control spectral data of a plurality of control media with known contents said control spectral data including data corresponding to multiple angles and/or wavelengths;
selecting one of said plurality of control media as a base media and labelling a corresponding spectral data as a base spectral data;
measuring a test spectral data of a test medium including data corresponding to said multiple angles and/or wavelengths;
determining a test ratio of a magnitude of the test spectral data to the base spectral data from said multiple angles and/or wavelengths; and
comparing the test ratio to ratios of magnitudes of a plurality of said control spectral data to said base spectral data to determine the contents of the test medium.

2. The method according to claim 1, further comprising mapping of the test spectral data and the base spectral data.

3. The method according to claim 2, further determining a ratio of the mapped test spectral data to the base spectral data.

4. The method according to claim 3, further comprising comparing the test ratio of the mapped spectral data to ratios of the control spectra in a database.

5. The method according to claim 2, wherein the map is a three-dimensional spectral map wherein a first axis is relative angle, a second axis is wavelength, and a third axis is intensity.

6. The method according to claim 1, further comprising collecting the spectral data by at least one detector.

7. The method according to claim 6, wherein the collecting is by at least one detector which is stationary relative to the test medium.

8. The method according to claim 6, wherein the collecting is by at least one detector which is rotating relative to the test medium.

9. The method according to claim 1, further comprising at least one light source configured for illuminating the test medium.

10. The method according to claim 1, where said comparing is via an artificial intelligence algorithm (AI) configured to process and analyze spectral data.

11. A system for detecting and identifying substances in fluid, comprising:
at least one light source configured for illuminating a fluid sample;
at least one optical detector configured for detecting light exiting or passing through the fluid sample, wherein the at least one optical detector is configured to detect light passing through the fluid sample at various angles to the at least one light source; and
a processor configured for collecting and processing spectral data for different wavelengths, different relative angles or both.

12. The system according to claim 11, wherein the processor is configured to control the relative angle between the light source and the detector.

13. The system according to claim 12, wherein the processor is configured to control the relative angle between the light source and the detector by:
a) selecting multiple stationary light sources and detectors; or
b) moving a detector or light source with an actuator; or
c) measuring light exiting or passing through the fluid sample from a stationary or moving light source at different angles with multiple stationary or moving detectors simultaneously; or
d) any combination of steps a, b and c.

14. The system according to claim 11, further comprising a database connected to the processor.

15. The system according to claim 11, wherein the at least one light source is stationary relative to the fluid sample.

16. The system according to claim 11, wherein the at least one light source is rotating relative to the sample.

17. The system according to claim 11, wherein the at least one detector is stationary relative to the fluid sample.

18. The system according to claim 11, wherein the at least one detector is rotating relative to the fluid sample.

19. The system according to claim 11, wherein the processor includes an artificial intelligence algorithm (AI) configured to process and analyze spectral data.

20. The system according to claim 11, wherein said processor is further configured for:
measuring control spectral data of a plurality of control media with known contents said control spectral data including data corresponding to multiple angles and/or wavelengths;
selecting one of said plurality of control media as a base media and labelling a corresponding spectral data as a base spectral data;
measuring a test spectral data of a test medium including data corresponding to said multiple angles and/or wavelengths;
determining a test ratio of a magnitude of the test spectral data to the base spectral data from said multiple angles and/or wavelengths; and
comparing the test ratio to ratios of magnitudes of a plurality of said control spectral data to said base spectral data to determine the contents of the test medium.
